(19) United States
(12) Patent Application Publication
(10) Pub. No.: US 2007/0173853 A1
(43) Pub. Date: Jul. 26, 2007

(54) System and Method for Centering Surgical Cutting Tools About the Spinous Process or Other Bone Structure

(75) Inventor: Michael MacMillan, Gainesville, FL (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: University of Florida Research Foundation, Inc.

(21) Appl. No.: 11/621,737

(22) Filed: Jan. 10, 2007

Related U.S. Application Data
(60) Provisional application No. 60/758,327, filed on Jan. 11, 2006.

(51) Int. Cl. A61F 5/00

(52) U.S. Cl. 606/87

(57) ABSTRACT

Various embodiments of the present invention provide, for example, a system and method for centering a surgical tool about the spinous process or other bony structure. Certain embodiments of the present invention may guide the surgical tool along a posterior midline of the spine in order to divide the spinous process. Various embodiments of the present invention may also further provide a system and method for performing a minimally invasive laminectomy procedure via the midline approach described above that may thus reduce the trauma experienced by tissues surrounding the spine or other bony structure.

### CROSS-REFERENCE TO RELATED APPLICATIONS

This application claims priority to U.S. Provisional Patent Application No. 60/758,327, filed Jan. 12, 2006, which is hereby incorporated by reference herein in its entirety.

### FIELD OF INVENTION

Various embodiments of the present invention relate to devices and methods for centering surgical cutting tools about a bony projection such as the spinous process. For example, some embodiments of the present invention may provide a centering method to better enable a minimally-invasive surgical procedure for splitting a spinous process at the dorsal midline of a subject in order to perform a spinal decompression procedure, such as laminectomy, for treating lumbar stenosis.

### BACKGROUND OF THE INVENTION

A key issue in the safe and effective performance of minimally-invasive surgical procedures that involve the cutting of bone (particularly the cutting of cortical bone making up portions of the spinal column) is the protection of critical and often sensitive areas of soft tissue that may surround and/or be encased within the bone structure. For example, conventional treatments for lumbar spinal stenosis, which is characterized by the compression of the spinal canal and the neural elements encased therein, include the removal and/or adjustment of bone structures (lamina) that encase the spinal canal. Such stensoses are the most common indication for surgery of the spine in patients over age 65.

Surgical approaches to the treatment of lumbar stenosis have the goal of decompressing the neural elements. This has been accomplished in conventional methods by the aggressive resection of the posterior bony elements of the spine via an extensile midline approach. Such treatments are often called “wide laminectomies” and, while often successful in decompressing the neural elements, the resection of bony structural elements in the spine, such as the pars interarticularis, facet joints, and the spinous processes, were found to often result in significant morbidity and iatrogenic instability.

Research on lumbar stenosis pathophysiology has indicated that the symptoms of lumbar stenosis result from a complex combination of facet arthropathy and hypertrophy, ligamentum flavum hypertrophy, invertebral disc bulging or herniation, and congenital narrowing of the spinal canal. Furthermore, advances in noninvasive imaging have shown that the majority of compression of the spinal canal occurred at the level of the interlaminar window. This discovery led to the application of laminotomies of the interlaminar windows or reconstructive laminoplasty to allow for decompression of the neural structures while also preserving posterior stabilizing structures. These types of conventional techniques have been used for decades with varying degrees of success.

Conventional “open” midline surgical procedures for treating lumbar spinal stenosis have continued to present problems for patients caused by dead space, local wound complications, and tissue trauma including denervation of the paraspinal musculature and subsequent atrophy. The extensive exposures required for adequate visualization when performing such “open” decompression techniques are associated with significant morbidities and complications. For example, several studies have confirmed that the most influential etiology in post-operative complications was tissue trauma and the subsequent stress response. Tissue trauma, pain, prolonged hospitalization, extended recovery, and medical complications related to the stress of duration of conventional midline “open” procedures have all been contributory to mixed medical outcomes.

Newer conventional techniques for treating lumbar spinal stenosis via decompression have centered on percutaneous, micro-endoscopic, and image-guided techniques in order to minimize tissue trauma by limiting the need for exposure. Such minimally-invasive procedures have become increasingly utilized in the treatment of a wide variety of diseases and conditions because of these benefits. The conventional surgical decompression procedures of laminectomy, laminotomy, and laminoplasty have been attempted via minimally-invasive procedures in order to minimize surgical trauma and decrease post-surgical morbidity. For example, microendoscopic decompressive laminotomy (MEDL) approaches have been used to treat lumbar spinal stenosis wherein surgical instruments are introduced via a unilateral transmuscular approach (wherein the endoscopic instruments travel through the paraspinal muscles on either side of the spinous process to reach the lamina). While these newer conventional techniques may reduce the overall exposure of spinal tissues and supporting structures, MEDL procedures are technically demanding and continue to result in problems including ipsilateral facet complex disruption, nerve root injury, and dural tear resulting from difficult visualization. In addition, difficult visualization and awkward working angles resulting from these conventional minimally-invasive unilateral approaches may also result in the inadequate decompression of the contralateral lateral recess or foramen.

Thus, there remains a need in the art for a minimally-invasive technique for treating lumbar stenosis that not only minimizes trauma on adjacent tissues but that also more reliably results in the decompression of the neural tissues. There also exists a need in the art for a system of specialized instruments for more reliably achieving an alternative minimally-invasive approach for treating lumbar stenosis via decompression that provides superior visualization of the relevant tissues and reduces the incidence of potentially damaging misalignment of surgical tools during the procedure.

### SUMMARY OF THE INVENTION

Various embodiments of the present invention satisfy the needs listed above and may provide other advantages as described below. Embodiments of the present invention may include a method for performing a minimally-invasive midline decompression procedure by dividing the spinous process along a posterior axis defined by the superior and inferior extents of the spinous process. According to some embodiments, the method comprises operably engaging a cutting guide device with a fascia surrounding the spinous process. Thus, the cutting guide device may be positioned substantially adjacent to the spinous process. Furthermore, the cutting guide device may define a cutting channel extending therethrough such that the spinous process is substantially accessible from a posterior position via the cutting channel. The method may further comprise inserting a cutting device into the cutting channel defined by the cutting guide device such that the cutting guide device directs the cutting device in an anterior direction and though the posterior axis of the spinous process so as to divide the spinous process into a right portion and a left portion substantially along the posterior axis.

According to other method embodiments of the present invention, the step for operably engaging the cutting guide device with the fascia may also comprise inserting a first alignment pin in the spinous process at a superior position along the posterior axis and inserting a second alignment pin in the spinous process at an inferior position along the posterior axis. Furthermore, some method embodiments may further comprise placing an inner guide device over the first and second alignment pins such that a major axis of the inner guide device is substantially parallel to the posterior axis and such that the inner guide device is substantially adjacent to the spinous process. According to various embodiments of the present invention, the inner guide device may define a central channel extending therethrough. Furthermore, the central channel defined in the inner guide device may have a superior end and an inferior end, wherein the superior end is configured to receive the first alignment pin and wherein the inferior end is configured to receive the second alignment pin. Various method embodiments of the present invention may also comprise surrounding the inner guide device with the cutting guide device so as to position the cutting guide device precisely relative to the stable position of the first and second alignment pins (which are inserted directly into the bone forming the spinous process, as described above). Furthermore, the cutting channel defined within the cutting guide device may be configured to be capable of receiving the inner guide device such that the major axis of the cutting guide device is substantially parallel to the posterior axis and such that the cutting guide device is substantially adjacent to the spinous process. In order to ensure the stability and steady position of the cutting guide device relative to the spinous process, the cutting guide device may also include an anterior side comprising a plurality of fascial penetration pins extending in an anterior direction substantially perpendicular to the anterior side for piercing the fascia so as to operably engage the cutting guide device with the fascia. Thus, according to some method embodiments, the cutting guide device may be substantially fixed relative to the spinous process via the engagement of the plurality of fascial penetration pins with the fascia surrounding the spinous process.

In order to clear the cutting channel of the cutting guide device, the method embodiments of the present invention may further comprise removing the first alignment pin, the second alignment pin, and the inner guide device from the spinous process such that the cutting channel is substantially open to receive and guide a cutting device in the anterior direction and though the posterior axis of the spinous process so as to divide the spinous process into a right portion and a left portion substantially along the posterior axis. According to some additional method embodiments, the method may further comprise retracting the right portion and the left portion of the spinous process to expose a laminar structure connected to and located substantially anterior to the spinous process.

Furthermore, in some method embodiments of the present invention directed specifically to laminectomy procedures and/or minimally invasive procedures for relieving lumbar stenosis, the method may further comprise removing the laminar structure from the right portion and the left portion of the spinous process so as to relieve a compressive force exerted by the laminar structure on a spinal canal located substantially anterior to the laminar structure. In some embodiments, the removing step described above may further comprise inserting a laminectomy tool between the right portion and the left portion of the spinous process. According to various embodiments of the present invention, the laminectomy tool may comprise a shaft portion, a handle portion extending substantially perpendicular from a posterior end of the shaft portion, and a blade portion extending substantially perpendicular from an anterior end of the shaft portion and substantially parallel to the handle portion. Thus, according to some such embodiments, a user may rotate the handle portion to correspondingly rotate the blade portion to remove the laminar structure from the right portion and the left portion of the spinous process.

Other embodiments of the present invention further comprise a system of interconnected and/or related devices for performing a minimally-invasive spinal surgical procedure via a spinous process defining a posterior axis. For example, according to some system embodiments of the present invention, the system may comprise: a first alignment pin for insertion in the spinous process at an superior position along the posterior axis; a second alignment pin for insertion in the spinous process at an inferior position along the posterior axis; and an inner guide device configured to be capable of operably engaging the first and second alignment pins such that a major axis of the inner guide device is substantially parallel to the posterior axis and such that the inner guide device is substantially adjacent to the spinous process. As described generally above with respect to the method embodiments of the present invention, the inner guide device may define a central channel extending therethrough, wherein the central channel includes a superior end and an inferior end. Furthermore, the superior end of the central channel may be configured to receive the first alignment pin and the inferior end may be correspondingly configured to receive the second alignment pin. The system may further comprise a cutting guide device defining a cutting channel extending therethrough. The cutting channel may be configured to be capable of receiving the inner guide device such that the major axis of the cutting guide device is substantially parallel to the posterior axis and such that the cutting guide device is substantially adjacent to the spinous process. As described generally above with respect to the method embodiments of the present invention, the cutting guide device may include an anterior side comprising a plurality of fascial penetration pins extending in an anterior direction substantially perpendicular to the anterior side for piercing the fascia. Thus, the cutting guide device may be configured to be capable of operably engaging the fascia so as to be substantially fixed relative to the spinous process such that when the inner guide device, the first alignment pin, and the second alignment pin are removed from the spinous process, the spinous process may be substantially accessible from a posterior position via the cutting channel defined in the cutting guide device.

Additional system embodiments of the present invention may further comprise a cutting device for dividing the spinous process into a right portion and a left portion substantially along the posterior axis. Furthermore, in some embodiments, the cutting device may be configured to be capable of being inserted through the cutting channel in the anterior direction and though the posterior axis of the spinous process. Other system embodiments may also comprise a laminectomy tool configured to be capable of being inserted between the right portion and the left portion of the spinous process. In some embodiments, the laminectomy tool may comprise: a shaft portion; a handle portion extending substantially perpendicular from a posterior end of the shaft portion; and a blade portion extending substantially perpendicular from an anterior end of the shaft portion and substantially parallel to the handle portion. Thus, according to some such system embodiments, a user may rotate the handle portion of the laminectomy tool to correspondingly rotate the blade portion to remove a laminar structure connected to an anterior portion of the spinous process. Because the cutting device and laminectomy tool are constrained within the cutting channel defined in the cutting guide device, system embodiments of the present invention may thus limit the angle at which the cutting device and/or laminectomy tool may be inserted through the divided portions of the spinous process, thereby limiting the chance that unintentional harm and/or undue trauma is experienced by the tissues surrounding the lamina and the spinous process.

Thus the various embodiments of the invention may provide certain advantages that may include, for example: addressing and effectively treating pathologies of the spine while reducing trauma on the surrounding spinal anatomy (including, for example, the paraspinal musculature and the supraspinous ligament); providing minimally-invasive access to the spinal canal for treating lateral disease; providing minimally-invasive access to the spinal canal for treating superior and/or inferior disease; providing minimally-invasive access to the spinal canal without violating surrounding muscular and/or nerve tissue; providing the opportunity for post-procedure healing via bone-to-bone lumbar fascia; and providing a minimally-invasive spine treatment that may obviate the need for spinal fusion procedures by preventing post-procedure lumbar instability.

These advantages, for example, and others that will be evident to those skilled in the art, may be provided in the various container method and system embodiments of the present invention for performing a minimally-invasive spinal surgical procedure via a spinous process.

### BRIEF DESCRIPTION OF THE DRAWINGS

Various embodiments of the invention will be better understood by reference to the Detailed Description of the Invention when taken together with the attached drawings, wherein:

FIG. 1 is a cross-sectional view of the spinous process and surrounding anatomy undergoing treatment via one embodiment of the method and system of the present invention wherein a cutting device is advanced to divide the spinous process;

FIG. 2 is a cross-sectional view of the spinous process and surrounding anatomy undergoing treatment via one embodiment of the method and system of the present invention wherein a laminotomy device is advanced to relieve a lumbar stenosis;

FIG. 3A is a perspective view of the posterior surface of an individual showing the projection of the spinous process along a posterior axis;

FIG. 3B is a perspective view of the posterior surface of an individual showing the projection of the spinous process defining a posterior axis and a pair of alignment pins inserted in superior and inferior positions along the posterior axis according to one embodiment of the present invention;

FIG. 3C is a perspective view of the posterior surface of an individual showing an inner guide device and a cutting guide device operably engaged about the spinous process via a pair of alignment pins according to one embodiment of the present invention;

FIG. 3D is a perspective view of the posterior surface of an individual showing the cutting guide device operably engaged with a fascia surrounding the spinous process after removal of the alignment pins and the inner guide device according to one embodiment of the present invention;

FIG. 4 is a detailed perspective view of the inner guide device operably engaged within the cutting channel of the cutting guide device according to one system embodiment of the present invention;

FIG. 5 is a detailed side view of an alignment pin according to one embodiment of the present invention;

FIG. 6A is a cross-sectional view of the spinous process and surrounding anatomy undergoing treatment via one embodiment of the method and system of the present invention wherein a laminectomy tool is advanced in the anterior direction between the right and left portions of the divided spinous process;

FIG. 6B is a cross-sectional view of the spinous process and surrounding anatomy undergoing treatment via one embodiment of the method and system of the present invention wherein a laminectomy tool is advanced and rotated to remove at least a portion of the lamina from the spinous process; and

FIG. 7 is a perspective view of a laminectomy tool according to one embodiment of the present invention.

### DETAILED DESCRIPTION OF THE INVENTION

Various embodiments of the present invention now will be described more fully hereinafter with reference to the accompanying drawings, in which some, but not all embodiments of the inventions are shown. Indeed, various embodiments of the inventions may be embodied in many different forms and should not be construed as limited to the embodiments set forth herein; rather, these embodiments are provided so that this disclosure will satisfy applicable legal requirements. Like numbers refer to like elements throughout. The singular forms “a,” “an,” and “the” include plural referents unless the context clearly dictates otherwise.

Although some embodiments of the invention described herein are directed to a method and system for performing a minimally-invasive spinal surgical procedure via a spinous process defining a posterior axis, it will be appreciated by one skilled in the art that the various embodiments of the invention are not so limited. For example, aspects of the cutting guide device, alignment pins, and other various embodiments of the present invention may also be used to center and establish “safe” cutting paths or axes through other bony structures that may be generally accessible to a clinician without the need for extensive surgical procedures. For example, certain of the various embodiments of the present invention may be used to center surgical cutting tools (such as a high-speed drill device) about the iliac crest for a bone graft harvest procedure, bone biopsy, and/or bone marrow harvesting procedure.

In addition, the alignment pins 110, 120 disclosed herein for fixing the cutting guide device 140 relative to the spinous process prior to commencement of the method for midline decompression described below may also be useful for establishing a dynamic reference arc for computer aided surgical techniques. For example, some forms of computer guided surgery require that a dynamic reference arc be rigidly attached to the anatomy of interest. Instruments then can be accurately guided to appropriate points on the patient in the area of the arc. With the advent of less invasive procedures, smaller incisions, and the percutaneous introduction of tools, there are fewer accessible anatomic structures onto which these dynamic reference arrays can be attached. Three anatomical locations on the lower trunk provide the possibility for rigid bony attachment: the posterior iliac crest, the anterior iliac crest and the spinous processes. Thus, alignment pins 110, 120 of the type described herein may be useful not only for placing the cutting guide device 140 described herein, but also for establishing a dynamic reference arc that may be attached to the embedded alignment pin(s) 110, 120 for the purpose of completing registration and guidance during computer-aided surgery (CAS).

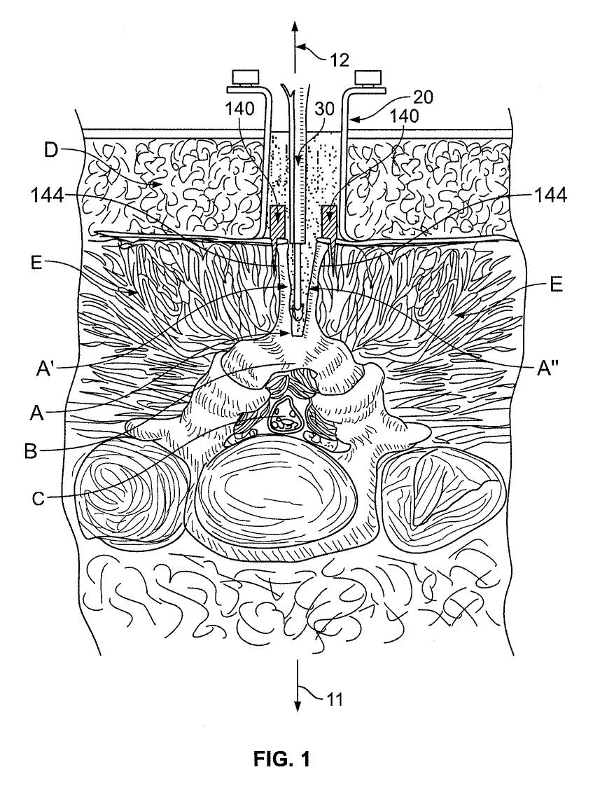

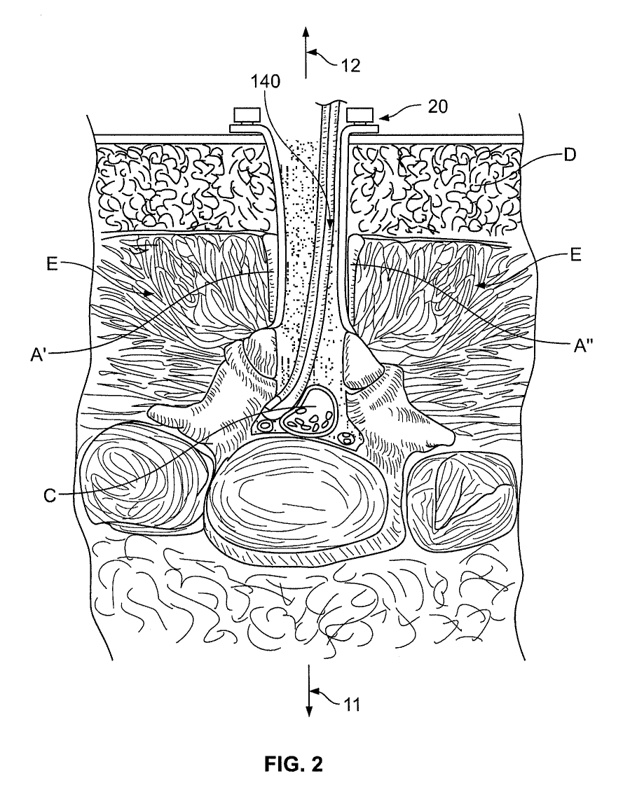

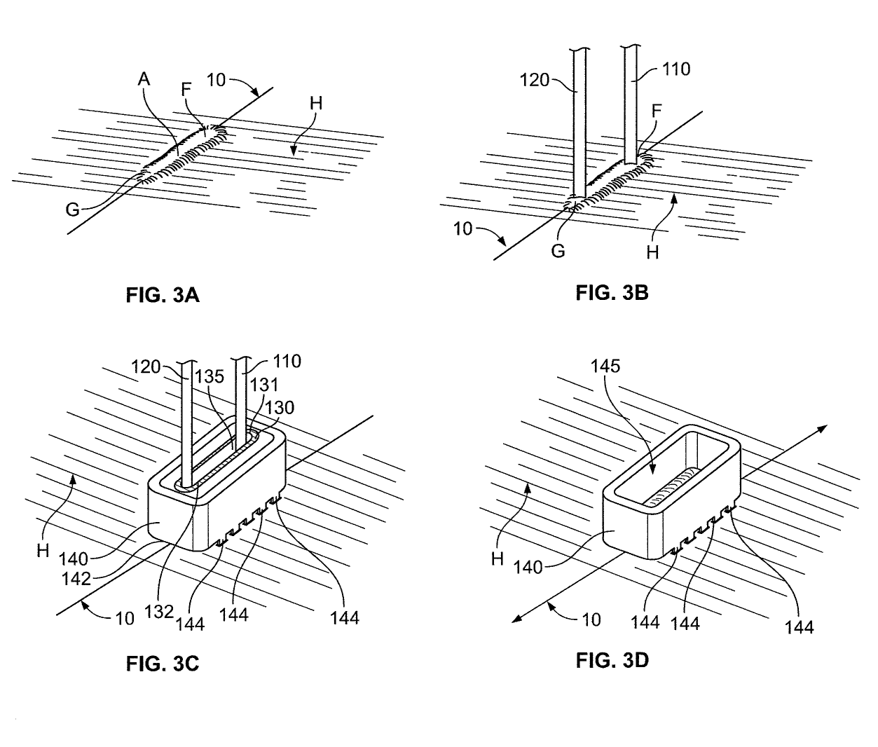

Embodiments of the present invention generally provide a method and system for performing a minimally-invasive spinal surgical procedure via a midline approach through the spinous process A defining a posterior axis 10. As shown generally in FIGS. 1 and 2, the lumbar vertebrae forming the inferior portion of the spine include a bony projection called the spinous process A that is generally visible through an individual's skin down the midline of the back (see generally FIG. 3A, showing the spinous process A as it appears projecting from an individual's posterior side). As summarized above, and as shown generally in FIG. 3, the present invention provides methods and systems for dividing the spinous process A along the posterior axis 10 into a left portion A′ and a right portion A″ such that the left and right portions A′, A″ may be retracted (using a retractor device 20, for example) to gain access to areas of the spinal anatomy that are connected to an anterior side of the spinous process A (including, for example, the lamina B surrounding the spinal canal C). Various embodiments of the present invention have a significant advantage over conventional minimally invasive spinal procedures that access the anterior 11 portion of the spinous process A and the lamina B via a cannula that is introduced laterally through the paraspinal musculature E, thereby inducing trauma on the paraspinal muscles E and the nerve tissue therein. The midline approach of the systems and method embodiments of the present invention thus has the advantage of avoiding the paraspinal musculature E that are positioned laterally about the posterior axis 10 defined by the spinous processes A.

However, in order to ensure a safe and accurate cutting path using the midline approach shown generally in FIGS. 1 and 2, the cutting device 30 must be accurately guided in the anterior direction 11 through the posterior axis 10 and into the spinous process A in order to divide the spinous process A into two generally mirrored portions A′, A″ that may be reattached post-procedure via bone-on-bone attachment techniques that will be appreciated by those skilled in the art. Thus, as shown generally in FIGS. 3A-3D embodiments of the present invention may provide a method and system for performing a minimally-invasive spinal surgical procedure via the spinous process A wherein the spinous process defines a posterior axis 10. As shown generally in FIG. 3D, one method embodiment of the present invention comprises operably engaging a cutting guide device 140 with a fascia H surrounding the spinous process A such that the cutting guide device 140 is substantially adjacent to the spinous process A. The cutting guide device 140 may define, for example, a cutting channel 145 extending therethrough such that the spinous process A is substantially accessible from a posterior position via the cutting channel 145.

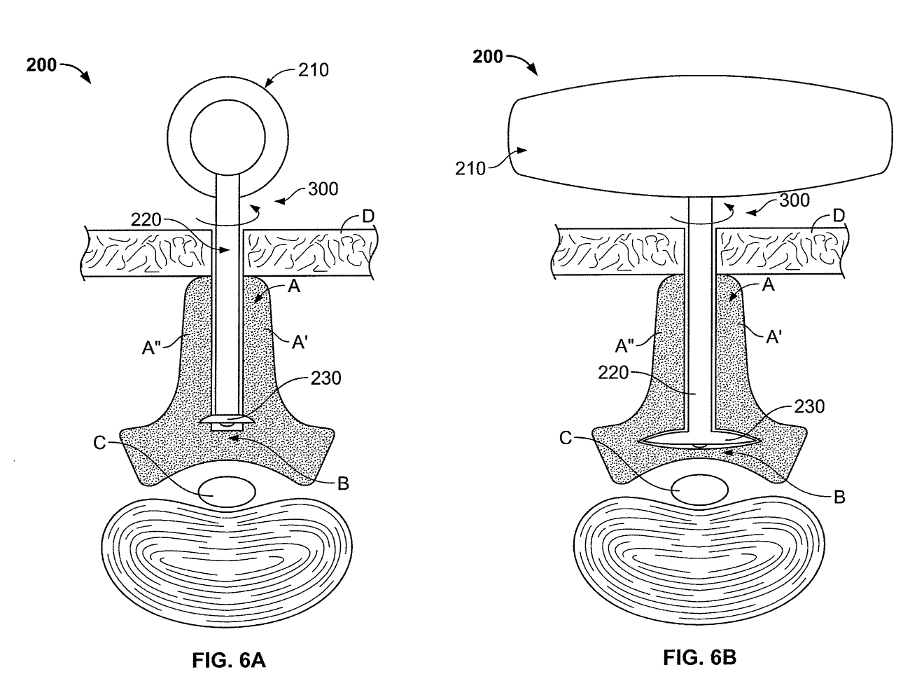

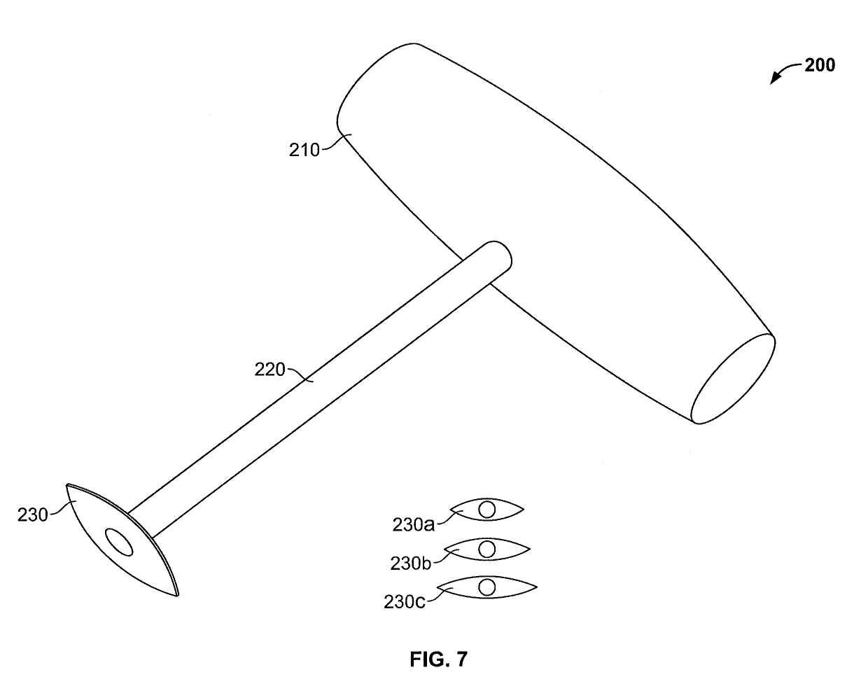

Method embodiments of the present invention may also comprise steps for inserting a cutting device 30 (such as a high-speed drill, for example, as shown generally in FIG. 1) into the cutting channel 145 defined by the cutting guide device 140 such that the cutting guide device 140 directs the cutting device 30 in an anterior direction 11 and though the posterior axis 10 of the spinous process A so as to divide the spinous process into a left portion A′ and a right portion A″ substantially along the posterior axis 10. As shown generally in FIG. 1, the cutting channel 145 defined by the cutting guide device 140 may be configured to receive a variety of microendoscopic tools that may include, but are not limited to: retraction devices 20, camera devices (for visualizing the fascia H surrounding the spinous process A, for example as well as critically sensitive tissues within the spinal canal C), cutting devices 30 (such as a high-speed drill for dividing the spinous process A as shown generally in FIG. 1), and laminectomy devices (such as the laminectomy tool 200 shown generally in FIGS. 6A, 6B, and 7). The relative height of the cutting guide device 140 may thus limit the range of angles at which the cutting device 30 enters the spinous process A so as to ensure that the cutting device 30 is advanced generally in the anterior direction 11 and is substantially centered in the spinous process A (as shown generally in FIG. 1, for example).

According to some method embodiments, as shown, for example in FIGS. 3A-3D, the step for operably engaging the cutting guide device 140 with the fascia H surrounding the spinous process A (described generally above) may further comprise inserting a first alignment pin 110 in the spinous process A at an superior position F along the posterior axis 10 and inserting a second alignment pin 120 in the spinous process A at an inferior position G along the posterior axis 10. According to some method embodiments, the alignment pins 110, 120 may be inserted into the spinous process A percutaneously. For example, a spinous process A is typically palpable underneath the skin and accessible for percutaneous pin placement wherein the insertion procedure first comprises the establishment of a stab incision over the desired spinous process A. Also, in some method embodiments, the top of the spinous process A may be palpated with a trocar (not shown) that may be introduced percutaneously within a cannula sleeve (not shown). For method embodiments of the present invention wherein the alignment pins 110, 120 are inserted into the spinous process A, the diameter of the trocar cannula may be relatively small, having inner diameters that may include but are not limited to a range from 3-5 millimeters. In addition, the trocar point may be short and pointed, with a 1-2 millimeter shoulder around the point to prevent excessively deep penetration into the structure of the spinous process A. Furthermore, to aid in the placement of the alignment pins 110, 120 in the spinous process A, the end of the outer cannula sleeve may be provided with two opposing curves carved out of the mouth of the cannula that form a “saddle” that can “ride” on top of the spinous process A. Once the cannula is positioned on top of the spinous process, the alignment pins 110, 120 may be introduced. According to some system embodiments of the present invention, the alignment pins 110, 120 (as shown, for example, in FIG. 5) may comprise a shaft diameter of 3-5 millimeters (corresponding, for example, to an inner diameter of the trocar used to introduce the alignment pin 110, 120. One embodiment of the alignment pins 110, 120 provided in the system of the present invention is shown, for example, in FIG. 5. The alignment pin 110 may comprise a generally circular shaft terminating at an anterior end portion 112 wherein the anterior end portion 112 is generally tapered to a rounded tip 115. Furthermore, the anterior end portion 112 may also comprise a tapered thread 114 extending radially outward therefrom such that the alignment pin may be introduced down the cannula and subsequently screwed in to the bony structure of the spinous process A to a depth of 20-30 millimeters. The depth of the alignment pin 110, 120 insertion may be monitored in some method embodiments of the present invention by lateral fluoroscopy of the spinal region, as will be appreciated by one skilled in the art. With the alignment pins 110, 120 in place along the posterior axis 10 of the spinous process A, the inner guide device 130 and cutting guide device 140 may be operably engaged with the fascia H surrounding the spinous process A. Furthermore, and as described generally above, the alignment pins 110, 120 may be alternatively used as fixed reference points for the establishment of a dynamic reference arc for a computer-guided surgical technique.

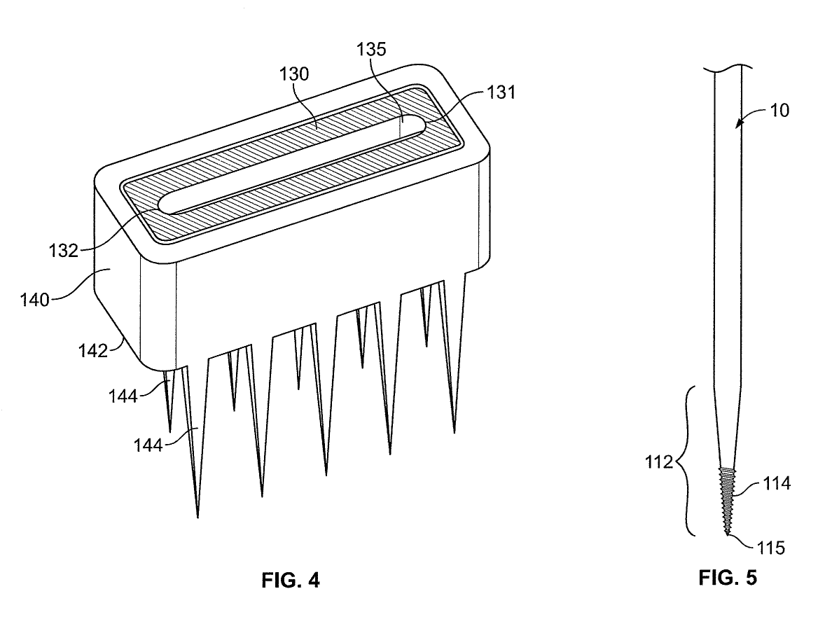

According to other embodiments, the step for operably engaging the cutting guide device 140 with the fascia H surrounding the spinous process A (described generally above) may further comprise placing an inner guide device 130 over the first and second alignment pins 110, 120 (as shown in FIG. 3C, for example) such that a major axis of the inner guide device 130 is substantially parallel to the posterior axis 10 and such that the inner guide device 130 is substantially adjacent to the spinous process A. Furthermore, according to the various system embodiments of the present invention, the inner guide device 130 may define a central channel 135 extending therethrough. The central channel 135 may further comprise a superior end 131 and an inferior end 132, wherein the superior end 131 is generally configured to receive the first alignment pin 110 and wherein the inferior end 132 is generally configured to receive the second alignment pin 120. Thus the inner guide device 130 may define a generally rectangular “footprint” posterior to the posterior axis 10 defined by the spinous process A so as to define an optimal area through which a cutting device 30 may be introduced in order to divide the spinous process A as shown generally in FIG. 1. Subsequent to the installation of the inner guide device 130 about the alignment pins 110, 120, some method embodiments of the present invention may further comprise surrounding the inner guide device 130 with the cutting guide device 140 (as shown generally in FIGS. 3C (showing the components of one system embodiment of the present invention installed in the fascia H) and FIG. 4 (showing the inner guide device 130 installed within the cutting channel 145 of the cutting guide device 140)). As shown in FIGS. 3C and 4, the cutting channel 145 defined by the cutting guide device 140 may be configured to be capable of receiving the inner guide device 130 such that the major axis of the cutting guide device 130 is substantially parallel to the posterior axis 10 and such that the cutting guide device 140 is substantially adjacent to the spinous process A. Furthermore, as shown in FIG. 4, the cutting guide device 140 may have an anterior side 142 comprising a plurality of fascial penetration pins 144 extending in an anterior direction 11 (see FIG. 1 showing the orientation of the fascial penetration pins 144 in the fascia H) substantially perpendicular to the anterior side 142. The fascial penetration pins 144 may thus effectively pierce the fascia H so as to operably engage the cutting guide device 140 with the fascia H so as to substantially fix the cutting guide device 140 relative to the spinous process A.

According to some method embodiments of the present invention, once the fascial penetration pins 144 are embedded in the fascia H surrounding the spinous process A (and the cutting guide device 140 is properly oriented by the cooperation of the cutting guide device 140 with the inner guide device 130 and the fixed alignment pins 110, 120), the method may further comprise removing the first alignment pin 110, the second alignment pin 120, and the inner guide device 130 from the spinous process A (as shown generally in FIG. 3D). Thus, the cutting channel 145 of the cutting guide device 140 may be left substantially open to receive and guide the cutting device 30 (and/or other microendoscopic devices) in the anterior direction 11 and though the posterior axis 10 of the spinous process A so as to divide the spinous process into a left portion A′ and a right portion A″ substantially along a plane extending in the anterior direction 11 from the posterior axis 10 defined by the spinous process A.

As shown generally in FIG. 2, the method embodiments of the present invention may further comprise steps for retracting the right portion A″ and the left portion A′ of the spinous process A to expose a laminar structure B connected to and located substantially anterior to the spinous process A. As one skilled in the art will appreciate, generally low-profile retracting devices may be used to expose the laminar structure B such that the portions A′, A″ of the spinous process A may more readily be re-attached post-procedure. According to some method embodiments of the present invention, the method for performing a minimally-invasive spinal surgical procedure via a spinous process may further comprise steps for treating a spinal stenosis such as, for example, a lumbar stenosis resulting in pressure being exerted on the spinal canal C by the lamina B. The spinous process A is generally continuous with the lamina B and generally arises in the posterior direction 12 from the lamina B. According to some such embodiments, the midline opening created by the division of the spinous process A (using the method embodiments of the present invention) may be suitable for performing a variety of minimally-invasive medical procedures that may include, but are not limited to: microendoscopic laminectomy; microendoscopic laminotomy; and microendoscopic foraminotomy. Therefore, in some method embodiments of the present invention, the method may further comprise steps for removing the laminar structure B from the right portion A″ and the left portion A′ of the spinous process A so as to relieve a compressive force exerted by the laminar structure B on a spinal canal C located substantially anterior to the laminar structure B (see FIG. 2 and FIGS. 6A-6B, for example). In some embodiments, the removing step described generally above may further comprise inserting a laminectomy tool 200 (such as a manual osteome as shown generally in FIG. 7) between the right portion A″ and the left portion A′ of the spinous process A. In some system embodiments of the present invention, the laminectomy tool 200 may comprise a powered and/or automated osteome device having a shaft of sufficient length to traverse the path from the posterior axis 10 of the spinous process A to the laminar structure B (as defined by, for example, the cutting device 30 and as maintained, for example, by the retractor device 20). According to other embodiments, the laminectomy tool 200 may further comprise a manual osteome as shown in FIGS. 6A, 6B, and 7 wherein the manual osteome comprises a shaft portion 220, a handle portion 210 extending substantially perpendicular from a posterior end of the shaft portion 220, and a blade portion 230 extending substantially perpendicular from an anterior end of the shaft portion 220 and substantially parallel to the handle portion 210 such that a user may rotate the handle portion 210 to correspondingly rotate the blade portion 230 to remove the laminar structure B from the right portion A″ and the left portion A′ of the spinous process A (as shown generally in FIGS. 6A and 6B (depicting an example of the cutting action exhibited by the manual osteome 200 embodiments of the present invention)). As shown in FIG. 7, various method embodiments of the present invention may involve the use of a laminectomy tool 200 (such as a manual osteome) having a blade portion 230 with a variety of different lengths that may be tailored to effectively perform a laminectomy, laminotomy, and/or microendoscopic foraminotomy procedure in individuals having spinous processes with varying geometries and/or sizes. As one skilled in the art will appreciate, a lateral fluoroscopy of the spinal region may aid in the determination of the width of the anterior portion of the spinous process A and/or the width of the interface between the spinous process A and the laminar structure B such that a clinician may choose an optimal size for the blade portion 230 of the laminectomy tool 200 during the course of the minimally-invasive procedure. For example, in some system embodiments of the present invention, the blade portion 230 may be provided with three standard lengths 230a, 230b, 230c that may be interchanged by the clinician prior to inserting the laminectomy tool 200 between the portions A′, A″ of the spinous process A. The lengths of the blade portion 230 of the laminectomy tool may include, but are not limited to: 10 millimeters, 13 millimeters, and 15 millimeters.

As described above, the various embodiments of the present invention also provide a system for performing a minimally-invasive spinal surgical procedure via a spinous process A defining a posterior axis 10. For example, as shown in FIG. 3B, some system embodiments may comprise a first alignment pin 110 for insertion in the spinous process A at an superior position F along the posterior axis 10 and a second alignment pin 120 for insertion in the spinous process A at an inferior position G along the posterior axis 10. As described generally above with respect to FIG. 5 the alignment pins 110, 120 may comprise a generally circular shaft terminating at an anterior end portion 112 wherein the anterior end portion 112 is generally tapered to a rounded tip 115. Furthermore, the anterior end portion 112 may also comprise a tapered thread 114 extending radially outward therefrom such that the alignment pin may be screwed into the bony structure of the spinous process A to a depth of, for example, 20-30 millimeters. The depth of the alignment pin 110, 120 insertion may be monitored in some method embodiments of the present invention by lateral fluoroscopy of the spinal region, as will be appreciated by one skilled in the art. With the alignment pins 110, 120 in place along the posterior axis 10 of the spinous process A, the inner guide device 130 and cutting guide device 140 may be operably engaged with the fascia H surrounding the spinous process A. Furthermore, and as described generally above, the alignment pins 110, 120 may be alternatively used as fixed reference points for the establishment of a dynamic reference arc for a computer-guided surgical technique.

The alignment pins 110, 120 may be composed of any suitable biocompatible material and preferably a biocompatible medical-grade metal alloy with a strength and hardness suitable for piercing and subsequently lodging in cortical bone structures such as the spinous process, iliac crest, or other hardened bony projection.

As shown in FIG. 3C, other system embodiments of the present invention may further comprise an inner guide device 130 configured to be capable of operably engaging the first and second alignment pins 110, 120 such that a major axis of the inner guide device 130 is substantially parallel to the posterior axis 10 and such that the inner guide device 130 is substantially adjacent to the spinous process A. Furthermore, the inner guide device 130 may define a central channel 135 extending therethrough having a superior end 131 configured to receive the first alignment pin 110 and an inferior end 132 configured to receive the second alignment pin 132. Thus, as shown in FIG. 3C, for example, the alignment pins 110, 120 may serve as a firm seat and reference point for placement of the inner guide device 130 posterior to the spinous process A. Furthermore, because the outer dimensions of the inner guide device 130 substantially correspond to the cutting channel dimensions of the cutting guide device 140 (described in detail below), the inner guide device may serve to center the cutting guide device 140 about the spinous process A such that the introduction of a cutting tool 30 (such as a high-speed drill, for example) though the cutting channel 145 will result in a substantially equal division of the spinous process A into a left portion A′ and a right portion A″ as shown generally in FIG. 1.

The inner guide device 130 may be composed of any suitable biocompatible material and in some cases, a biocompatible medical-grade engineering polymer with a strength and hardness suitable for receiving the alignment pins 110, 120 and maintaining a relatively rigid “footprint” for placing the cutting guide device 140 (described below) in a position immediately adjacent to and preferably centered about a posterior axis 10 defined by the spinous process A.

As summarized above, and shown in FIG. 3C, the system embodiments of the present invention may further comprise a cutting guide device 140 defining a cutting channel 145 extending therethrough. The cutting channel 145 defined by the cutting guide device 140 may be configured to be capable of receiving the inner guide device 130 such that the major axis of the cutting guide device 140 is substantially parallel to the posterior axis 10 and such that the cutting guide device 140 is substantially adjacent to the spinous process A. Furthermore, in some embodiments, the cutting guide device 140 may comprise an anterior side 142 from which a plurality of fascial penetration pins 144 may extend in an anterior direction 11 substantially perpendicular to the anterior side 142 for piercing the fascia H (surrounding the posterior portion of the spinous process A) so as to operably engage the cutting guide device 140 with the fascia H and so as to substantially fix the cutting guide device 140 relative to the spinous process A such that when the inner guide device 130, the first alignment pin 110, and the second alignment pin 120 are removed from the spinous process A (see FIG. 3D, for example), the spinous process A may be substantially accessible from a posterior position via the cutting channel 145. Therefore, the system embodiments of the present invention may advantageously establish a “safe” and substantially straight pathway in the anterior direction such that a cutting device 30 may be advanced in the anterior direction 11 and through the spinous process A so as to divide the spinous process into left and right portions A′, A″ as shown generally in FIG. 1.

The cutting guide device 140 may be composed of any suitable biocompatible material and in some cases, a biocompatible medical-grade engineering polymer with a strength and hardness suitable for maintaining a relatively rigid cutting channel 145 for placing the cutting guide device 140 (described below) in a position immediately adjacent to and preferably centered about a posterior axis 10 defined by the spinous process A. Furthermore, the fascial penetration pins 144 extending in the anterior direction from the anterior surface 142 of the cutting guide device 140 may be composed of a variety of biocompatible medical-grade metallic alloys with a strength and hardness suitable for piercing the fascia H and fixing the cutting guide device 140 in place relative to the spinous process A. In some system embodiments of the present invention, the fascial penetration pins 144 may be embedded within and/or otherwise operably engaged with the material making up the body of the cutting guide device 140. According to other embodiments such as, for example, in system embodiments wherein the cutting guide device 140 is composed of a metallic alloy or other metal material, the fascial penetration pins 144 may be formed as integral extensions of the cutting guide device 140. For example, in some embodiments, the entire cutting guide device 140 (including the fascial penetration pins 144 extending therefrom) may be machined from a single block of metal and/or polymeric material stock.

As shown in FIG. 1, some system embodiments of the present invention may further comprise a cutting device 30 for dividing the spinous process A into a right portion A″ and a left portion A′ substantially along the posterior axis 10. As described above with respect to the method embodiments of the present invention, the cutting device 30 may be configured to be capable of being inserted through the cutting channel 145 defined by the cutting guide device 140 in the anterior direction 11 and though the posterior axis 10 of the spinous process A. According to various embodiments of the present invention, the cutting device 30 may comprise a variety of manual and/or powered cutting implements suitable for cutting and/or dividing the spinous process A as shown generally in FIG. 1. For example, the cutting device 30 may comprise a high speed drill comprising a rotatable cutting bit having an outer diameter smaller than a width of the cutting channel 145 defined by the cutting guide device 145. In other system embodiments of the present invention, the cutting device 130 may comprise a number of different microendoscopic cutting tools and/or conventional surgical cutting tools that may include, but are not limited to: hydro-jet scalpels, reciprocating bone saws, manual saws, scalpels, drills, and/or combinations of the above-listed surgical tools.

Furthermore, and as shown generally in FIGS. 6A, 6B, and 7, some system embodiments of the present invention may further comprise a laminectomy tool 200 configured to be capable of being inserted between the right portion A″ and the left portion A′ of the spinous process A. In some embodiments, as shown in FIG. 7, the laminectomy tool 200 may comprise a manual osteome comprising a shaft portion 220, a handle portion 210 extending substantially perpendicular from a posterior end of the shaft portion 220, and a blade portion 230 extending substantially perpendicular from an anterior end of the shaft portion 220 and substantially parallel to the handle portion 210. Thus, according to such embodiments, and as shown generally in FIGS. 6A and 6B, a user may rotate the handle portion 210 to correspondingly rotate the blade portion 230 of the laminectomy tool 200 to remove a laminar structure B connected to an anterior portion of the spinous process A.

As shown in FIG. 7, various system embodiments of the present invention may further comprise a laminectomy tool 200 (such as a manual osteome) having a blade portion 230 with a variety of different lengths that may be tailored to effectively perform a laminectomy, laminotomy and/or microendoscopic foraminotomy procedure in individuals having spinous processes with varying geometries and/or sizes. As one skilled in the art will appreciate, a lateral fluoroscopy of the spinal region may be used to determination of the width of the anterior portion of the spinous process A and/or the width of the interface between the spinous process A and the laminar structure B such that a clinician may choose an optimal size for the blade portion 230 of the laminectomy tool 200 during the course of the minimally-invasive procedure. For example, in some system embodiments of the present invention, the laminectomy tool 200 may comprise several selectable blade portions 230 that may be selectively operably engaged with an anterior end of the shaft portion 220. Thus a clinician may select and utilize one of a plurality standard blade portion 230 lengths 230a, 230b, 230c that may be interchanged by the clinician prior to inserting the laminectomy tool 200 between the portions A′, A″ of the spinous process A (as shown generally in FIGS. 6A and 6B.

Many modifications and other various embodiments of the inventions set forth herein will come to mind to one skilled in the art to which these inventions pertain having the benefit of the teachings presented in the foregoing descriptions and the associated drawings. Therefore, it is to be understood that the various embodiments of the invention are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method for performing a minimally-invasive spinal surgical procedure via a spinous process defining a posterior axis, the method comprising:
operably engaging a cutting guide device with a fascia surrounding the spinous process such that the cutting guide device is substantially adjacent to the spinous process, the cutting guide device defining a cutting channel extending therethrough such that the spinous process is substantially accessible from a posterior position via the cutting channel;
inserting a cutting device into the cutting channel defined by the cutting guide device such that the cutting guide device directs the cutting device in an anterior direction and though the posterior axis of the spinous process so as to divide the spinous process into a right portion and a left portion substantially along a plane extending in the anterior direction from the posterior axis.

2. The method according to claim 1, wherein the operably engaging step further comprises:
inserting a first alignment pin in the spinous process at a superior position along the posterior axis; and
inserting a second alignment pin in the spinous process at an inferior position along the posterior axis, the first and second alignment pins being configured to align the cutting guide device with the posterior axis of the spinous process.

3. The method according to claim 2, further comprising attaching a reference arc to at least one of the first alignment pin and the second alignment pin, the reference arc being configured to position an instrument relative to the spinous process for a computer-assisted surgical procedure.

4. The method according to claim 2, wherein the operably engaging step further comprises:
placing an inner guide device over the first and second alignment pins such that a major axis of the inner guide device is substantially parallel to the posterior axis and such that the inner guide device is substantially adjacent to the spinous process, the inner guide device defining a central channel extending therethrough, the central channel having a superior end and an inferior end, the superior end being configured to receive the first alignment pin and the inferior end being configured to receive the second alignment pin;
surrounding the inner guide device with the cutting guide device, the cutting channel thereof configured to be capable of receiving the inner guide device such that the major axis of the cutting guide device is substantially parallel to the posterior axis and such that the cutting guide device is substantially adjacent to the spinous process, the cutting guide device comprising an anterior side comprising a plurality of fascial penetration pins extending in an anterior direction substantially perpendicular to the anterior side for piercing the fascia so as to operably engage the cutting guide device with the fascia and so as to substantially fix the cutting guide device relative to the spinous process;
removing the first alignment pin, the second alignment pin, and the inner guide device from the spinous process such that the cutting channel remains substantially open to receive and guide the cutting device in the anterior direction and though the posterior axis of the spinous process so as to divide the spinous process into a right portion and a left portion substantially along the posterior axis.

5. The method according to claim 1, further comprising:
retracting the right portion and the left portion of the spinous process to expose a laminar structure connected to and located substantially anterior to the spinous process.

6. The method according to claim 5, further comprising:
removing the laminar structure from the right portion and the left portion of the spinous process so as to relieve a compressive force exerted by the laminar structure on a spinal canal located substantially anterior to the laminar structure.

7. The method according to claim 6, wherein the removing step comprises inserting a laminectomy tool between the right portion and the left portion of the spinous process, the laminectomy tool comprising a shaft portion, a handle portion extending substantially perpendicular from a posterior end of the shaft portion, and a blade portion extending substantially perpendicular from an anterior end of the shaft portion and substantially parallel to the handle portion such that a user may rotate the handle portion to correspondingly rotate the blade portion to remove the laminar structure from the right portion and the left portion of the spinous process.

8. A system for performing a minimally-invasive spinal surgical procedure via a spinous process defining a posterior axis, the system comprising:
a first alignment pin for insertion in the spinous process at a superior position along the posterior axis;
a second alignment pin for insertion in the spinous process at an inferior position along the posterior axis;
an inner guide device configured to be capable of operably engaging the first and second alignment pins such that a major axis of the inner guide device is substantially parallel to the posterior axis and such that the inner guide device is substantially adjacent to the spinous process, the inner guide device defining a central channel extending therethrough, the central channel having a superior end and an inferior end, the superior end being configured to receive the first alignment pin and the inferior end being configured to receive the second alignment pin;
a cutting guide device defining a cutting channel extending therethrough, the cutting channel being configured to be capable of receiving the inner guide device such that the major axis of the cutting guide device is substantially parallel to the posterior axis and such that the cutting guide device is substantially adjacent to the spinous process, the cutting guide device comprising an anterior side comprising a plurality of fascial penetration pins extending in an anterior direction substantially perpendicular to the anterior side for piercing the fascia so as to operably engage the cutting guide device with the fascia and so as to substantially fix the cutting guide device relative to the spinous process such that when the inner guide device, the first alignment pin, and the second alignment pin are removed from the spinous process, the spinous process may be substantially accessible by a from a posterior position via the cutting channel.

9. The system according to claim 8, further comprising a cutting device for dividing the spinous process into a right portion and a left portion substantially along the posterior axis, the cutting device being configured to be capable of being inserted through the cutting channel substantially along a plane extending in the anterior direction from the posterior axis.

10. A system according to claim 9, further comprising a laminectomy tool configured to be capable of being inserted between the right portion and the left portion of the spinous process.

11. A system according to claim 10, wherein the laminectomy tool comprises a shaft portion, a handle portion extending substantially perpendicular from a posterior end of the shaft portion, and a blade portion extending substantially perpendicular from an anterior end of the shaft portion and substantially parallel to the handle portion such that a user may rotate the handle portion to correspondingly rotate the blade portion to remove a laminar structure connected to an anterior portion of the spinous process.

12. A system according to claim 8 further comprising a reference arc configured to be operably engaged with at least one of the first alignment pin and the second alignment pin, the reference arc being configured to position an instrument relative to the spinous process for a computer-assisted surgical procedure.

13. A system according to claim 9, wherein the cutting device is selected from the group consisting of:
hydro-jet scalpels;
reciprocating bone saws;
manual saws;
scalpels;
drills; and
combinations thereof.
